(19) United States
(12) Patent Application Publication
(10) Pub. No.: US 2023/0070622 A1
(43) Pub. Date: Mar. 9, 2023

(54) MEDICAL, DENTAL, OR VETERINARY DEVICE WITH USE INDICATOR

(71) Applicant: SEIDY FIGUEROA, BURBANK, CA (US)

(72) Inventor: SEIDY FIGUEROA, BURBANK, CA (US)

(21) Appl. No.: 17/901,499

(22) Filed: Sep. 1, 2022

Related U.S. Application Data
(60) Provisional application No. 63/239,867, filed on Sep. 1, 2021.

(51) Int. Cl. A61B 10/00; G01N 33/84; A61L 2/26

(52) U.S. Cl. CPC A61B 10/0045; G01N 33/84; A61L 2/26

(57) ABSTRACT

A medical, dental, or veterinary device with a use indicator includes a proximal end and a distal end configured to interact with a human or animal subject, the distal end including a use indication portion, wherein the use indication portion includes a reactive agent to react with a foreign substance to exhibit a visible change in appearance of the reactive agent to indicate to a veterinary, dental, or medical professional that the device had been previously used and must be sterilized or disposed.

### CROSS-REFERENCE

This application claims the benefit of U.S. provisional application No. 63/239,867, filed Sep. 1, 2021, the content of which is incorporated herein in its entirety.

### TECHNICAL FIELD

The disclosed technology relates generally to medical, dental, or veterinary devices, and more specifically some embodiments relate to systems and methods for preventing the unintentional reuse of medical, dental, or veterinary devices.

### BACKGROUND

Medical, dental, and veterinary devices are often made to be disposable and shipped in sterile packaging to avoid contamination. Such devices, once used, become exposed to contaminants such as bodily fluids, bacteria, and viruses. Those disposable devices should then be discarded. However, in some instances, medical, dental, and/or veterinary personnel may become confused and unintentionally reuse the device, thinking that it had yet to be used. Such mistakes lead to the unintentional spreading of contaminants, and could lead to infection and/or other complications.

For example, with the recent COVID-19 pandemic, demand for intubation during hospitalizations has increased. Part of the intubation process involves the evacuation of bodily secretions (e.g., saliva, blood, phlegm, etc.). Devices used to perform those procedures may have selectively detachable suction tips, wherein the selectively detachable suction tips are disposable and intended for one-time use. Limiting the disposable suction tips to one-time use reduces instances of cross-contamination between patients and inhibits the spread of infectious disease.

Currently, unused disposable suction tips can be confused with used disposable suction tips in cases where evacuated material happens to not collect visibly in the disposable suction tip. In instances where confusion occurs, likelihood of reuse of a previously used disposable suction tip is increased. When a disposable suction tip is reused, bodily secretions can be carried from one patient to the next, creating unnecessary opportunities for the spread of infectious disease.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1A illustrates a medical suction tip combined with a use indicator, consistent with embodiments disclosed herein.

FIG. 1B illustrates a perspective view of a medical suction tip combined with a use indicator, consistent with embodiments disclosed herein.

FIG. 2 illustrates a medical suction tip combined with a use indicator as it is used in a medical procedure wherein bodily secretions are evacuated from the patient, consistent with embodiments disclosed herein.

FIG. 3 is a flowchart describing a protocol for the prevention of unintentional reuse of disposable medical devices, consistent with embodiments disclosed herein.

FIG. 4 is a decision tree depicting a generalized procedure for preventing the unintended reuse of medical, dental, or veterinary devices, consistent with embodiments disclosed herein.

FIG. 5 illustrates a diagram of a computer system, consistent with embodiments disclosed herein.

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION OF THE EMBODIMENTS

Embodiments disclosed herein are directed to devices designed for used for medical, dental, or veterinary procedures. More specifically, some embodiments disclosed herein describe systems and procedures designed to reduce incidences of unintentional reuse of such devices. Unintentional reuse of such devices is often caused by confusion by medical, dental, or veterinary professionals regarding whether or not a device has been previously used. Such unintentional reuse may spread contaminants to patients, such as bacteria, virus, body fluids, or other contaminants. Components are disclosed which may be combined with said devices to better indicate whether or not a device has been previously used. These components, herein described as use indicators, signal whether the device has been previously used by reacting with features commonly found in the bodily environment in which they are intended to be exposed, or by reacting to the manipulation of the medical, dental, or veterinary professionals using the associated devices.

While the disclosure herein often describes a use indicator in combination with a medical suction tip being used to evacuate bodily secretions from a patient's respiratory pathway, upon reading this disclosure a person of ordinary skill in the art will readily understand that a use indicator may be combined with numerous medical, dental, or veterinary devices intended for insertion into the body of a patient, including non-disposable devices that need to be sterilized after each use. In applications not involving the human respiratory tract, the indicator device may be altered to react to the common properties of the area of the body where the medical, dental, or veterinary device is intended to be inserted.

Protocols and procedures are disclosed herein where medical, dental, and veterinary professionals check the associated use indicators before utilizing the associated device in an operation. With the adoption of such protocols and procedures, instances of confusion between used and unused medical, dental, and veterinary devices will likely be reduced. This reduction in confusion will likely lead to reduced instances of unintentional reuse of medical, dental, and veterinary devices. This reduction in unintentional reuse of medical, dental, and veterinary devices will likely lead to reduced instances where unsanitary objects are unnecessarily introduced during medical, dental, and veterinary procedures. This reduction in introductions of unsanitary objects will in turn likely lead to a stemming of the spread of infectious disease, and more sanitary medical, dental, and veterinary procedures overall.

In some embodiments, the use indicator may include a substrate (e.g., a chemically treated paper strip) that reacts with ions in a liquid solution or suspension (e.g., blood, saliva, urine, mucus, or other body fluids) and alters in visual appearance based on the pH of the liquid solution or suspension (e.g., a pH indicator strip). In some embodiments, the use indicator may include a substrate that reacts with and changes appearance in response to moisture, for example, to detect humidity from a respiratory tract or body fluid. In some embodiments, the use indicator may include a substrate that reacts with and changes appearance in response to hormones suspended in body fluid or aerosolized.

It should be understood that the word “reaction” as used herein is not limited to chemical reactions, and may include many other types of reactions. In some embodiments, a mechanical reaction may be used (e.g. a change in appearance after physical compression or increased heat caused by contact with body fluid or living organic material, or caused by manipulation from the medical, dental, or veterinary professional). In some embodiments, upon reorientation of the combined device, an enclosed ball rolls into a slot, indicating that the combined device has previously taken an orientation suggestive of use.

In some embodiments, the device may be packaged in a vacuum sealed package, and the opening of the package exposes the use indicator to air, and the use indicator may react with and change appearance in response to moisture or chemicals present in the air, such as nitrogen, oxygen, or carbon dioxide.

In some embodiments, the packaging may be coupled to the use indicator, and dislodging the use indicator from the packaging may trigger a reaction and appearance change on the use indicator.

In some embodiments, computing devices and software may be employed on the use indicator itself or on a separate computer system in order to utilize sensors or database systems. In embodiments where computing devices and software are employed to utilize database systems, a medical, dental, or veterinary professional interacts with the use indicator or a separate computer system in order to catalogue each use of an associated device and relate it to the exact device used. Medical, dental, or veterinary professionals wishing to later use a device then interact with the device or a computer to determine whether the device has been used previously.

Generally, any reaction which results in a detectable and/or conspicuous change in the use indicator after contact with the patient being operated on, or after contact with the medical, dental or veterinary professional using the device, will suffice as a mechanism for use indication. For example, the color of the use indicator may change.

Many of the figures disclosed herein depict a use indicator as combined with a disposable medical suction tip, e.g., a Yankauer suction tip. The use indicator may also be incorporated on other medical, dental, or veterinary devices intended for contact with and/or insertion into bodily environments. The use indicator may be combined with disposable or non-disposable devices, and used with medical, dental, or veterinary devices.

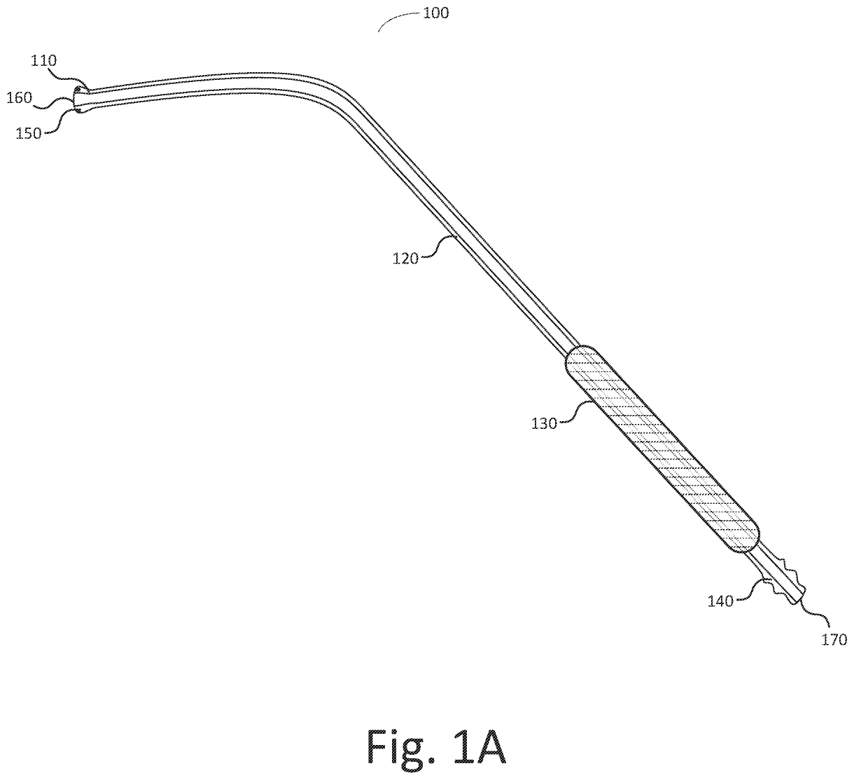

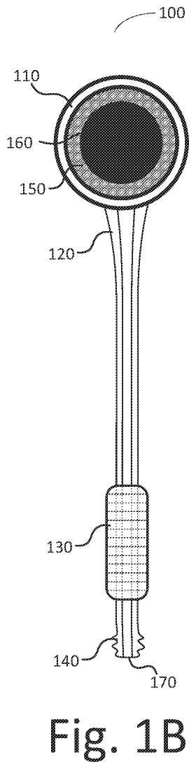

FIG. 1A illustrates an example medical suction tip (e.g., a Yankauer tip) combined with a use indicator, consistent with embodiments disclosed herein. The medical suction tip 100 has a distal end 110, a rigid tubing 120, a proximal end 130, a grooved connecting interface 140, a use indicator 150, an inlet 160, and an outlet 170.

The distal end 110 may be formed into a bulbous tip, presenting dull edges to the walls of the bodily cavity in which the medical suction tip 100 is inserted. These dull edges reduce risk of damaging the walls of the bodily cavity while the inlet 160 of the rigid tubing 120 is imposed against the walls of the bodily cavity in order to suck in various bodily secretions.

The use indicator 150 may be disposed on the distal end 110 of the medical suction tip 100, preferably as close as possible to the inlet 160 of the rigid tubing 120. Such placement may result in insertion of the use indicator 150 into the bodily cavity upon any use of the medical suction tip 100. The use indicator 150 may include a substrate that chemically reacts with moisture present in the bodily cavity and responds with a visible change in appearance (e.g., change in color or translucence). The appearance change may be irreversible, such that the appearance will not revert to an the initial state after drying. In some examples, the appearance change may include a display of a pattern, shape, or other observable physical property. When a user (e.g., a medical, dental, or veterinary professional) intending to reuse the same medical suction tip 100, the user will see that the observable physical property of the use indicator 150 has changed in appearance to indicate that the device has already been used, and the user may infer that the medical suction tip 100 has been previously used. If the medical suction tip 100 is disposable, then the user may dispose of the current medical suction tip 100 in favor of an unused one. If the medical suction tip 100 is non-disposable, then the user should sterilize the medical suction tip 100 before attempting to reuse it, and replace the spent use indicator 150.

The distal end 110 can alternatively be shaped in any way as demanded by the particular application of the medical procedure. For instance, in the context of medical suction tips, the distal end 110 could instead be shaped in a way to create a Poole or Frazier suction tip, or any other commonly known suction tip formation. In other formations, the use indicator 150 may be placed in a different locality in order to increase the likelihood that the use indicator 150 will be inserted into the bodily environment or exposed to body fluids when the medical suction tip 100 is used.

The rigid tubing 120 may run the entire length of the medical suction tip 100. The rigid tubing 120 preserves vacuum suction introduced in the medical suction tip 100 and conveys bodily secretions sucked into the inlet 160 through the medical suction tip 100 to outlet 170. The proximal end 130 of the medical suction tip 100 is depicted herein as including an optional textured area for easier handling by the user operating the medical suction tip 100. The rigid tubing 120 may terminate with a grooved connecting interface 140 near the outlet 170. The grooved connecting interface 140 may provide a grooved surface to which a flexible hose may be coupled. In one embodiment, a flexible hose leading to a medical waste container and a vacuum pump may be coupled to the grooved connecting interface 140. The vacuum pump imposes a vacuum across the rigid tubing 120, which in turn allows suction to be imposed at the inlet 160. The suction at the inlet 160 causes bodily secretions to be sucked into the rigid tubing 120 and conveyed through the medical suction tip 100 down to the outlet 170 and into the flexible hose. The flexible hose then conveys the evacuated bodily secretions down into a medical waste container.

FIG. 1B illustrates a perspective view of a medical suction tip combined with a use indicator, consistent with embodiments disclosed herein. This alternate perspective view of the medical suction tip 100 focuses on the shape and orientation of the use indicator 150. The distal end 110, rigid tubing 120, proximal end 130, grooved connecting interface 140, inlet 160, and outlet 160 are kept in view to better illustrate how the use indicator 150 is positioned relative to the rest of the components of the medical suction tip 100.

The use indicator 150 is depicted herein as a circular strip of material placed on the distal end 110 of the medical suction tip 100, around the inlet 160. Placing the use indicator 150 near the inlet 160 helps increase the likelihood that the use indicator 150 will be exposed to the bodily environment along with the medical suction tip 100 upon use of the medical suction tip 100. The inlet 160 is inserted on use of the medical suction tip 100 because the inlet 160 is where bodily secretions are sucked into the medical suction tip 100 for extraction along the rigid tubing 120, out of the outlet 170. Shaping and placing the use indicator 150 around the inlet 160 results in numerous contacts between the use indicator 150 and the bodily environment in which the medical suction tip is being used (e.g. the patient's respiratory pathway). When the use indicator 150 makes contacts with moisture, body fluid, and/or organic material in the bodily environment, the materials of the use indicator 150 may react with the moisture, body fluid, and/or organic material, resulting in a change in color, pattern, shape, or any other easily observable physical property of the use indicator 150. If the reaction is permanent or semi-permanent, the medical professional operating the medical suction tip 100 can inspect the use indicator 150 before any use in order to discern if the medical suction tip 100 has been previously used.

Though it is depicted herein as a strip of reactive material placed near the inlet, it should be understood that the use indicator 150 can take numerous positions on the combined device. For example, the use indicator 150 could instead take the form of a block of material, sitting further back on the tubing 120 in order to make contact with a portion of the bodily environment more unique or readily reactive, and therefore more indicative of use. As another example, the use indicator could be located on or near the handle 130, and instead be designed to react with the medical professional's hand as the medical professional grips the handle.

Numerous reactions may be employed to achieve the indication of use. In one embodiment, the use indicator 150 may be a pH indicator (e.g., Litmus paper), selected to change color upon contact with a solution or suspension and change appearance to indicate a pH value or values (for example, that may indicate the pH of body fluid). In another embodiment, the use indicator 150 may be a humidity indicator, selected to change color in response to moisture in the air (e.g., inside a body orifice). In another embodiment, the use indicator 150 may be a strip of paper inscribed with symbols, wherein the strip of paper reacts with hormones and/or antigens present in body fluid or cellular material within a body orifice.

It should be understood that the reaction type is not limited to chemical reactions, and may include many other types of reactions. In some embodiments, a mechanical reaction may be used. For instance, upon physical compression of the use indicator, the indicator may permanently change its appearance. In some embodiments, the removal of the indicator from packaging may trigger an appearance change by removal of a sticker, paper insert, and/or exposure to air. In some examples, upon horizontal orientation of the combined device, an enclosed ball may roll into a slot, indicating that the combined device has previously taken an orientation suggestive of use. In some embodiments, upon a flow of heat representative of an operator's hand or a patient's bodily environment, a material may chemically or physically react, resulting in conspicuous changes to the use indicator's color, pattern, shape.

Reactions implemented in software on computing devices may also be used to implement a use indicator 150. In some embodiments, a digital logging and/or RFID systems may be employed, with the associated database being stored either on the use indicator 150 itself, or on a separate computer system. In such embodiments, the medical, dental, or veterinary professional may update the database manually, or the database may be automatically updated upon the triggering of an electronic or mechanical sensor embedded on the use indicator 150.

Generally, any reaction which results in a detectable change in the use indicator 150 upon contact with the intended environment within which the combined device is to be used will suffice as a mechanism for use indication.

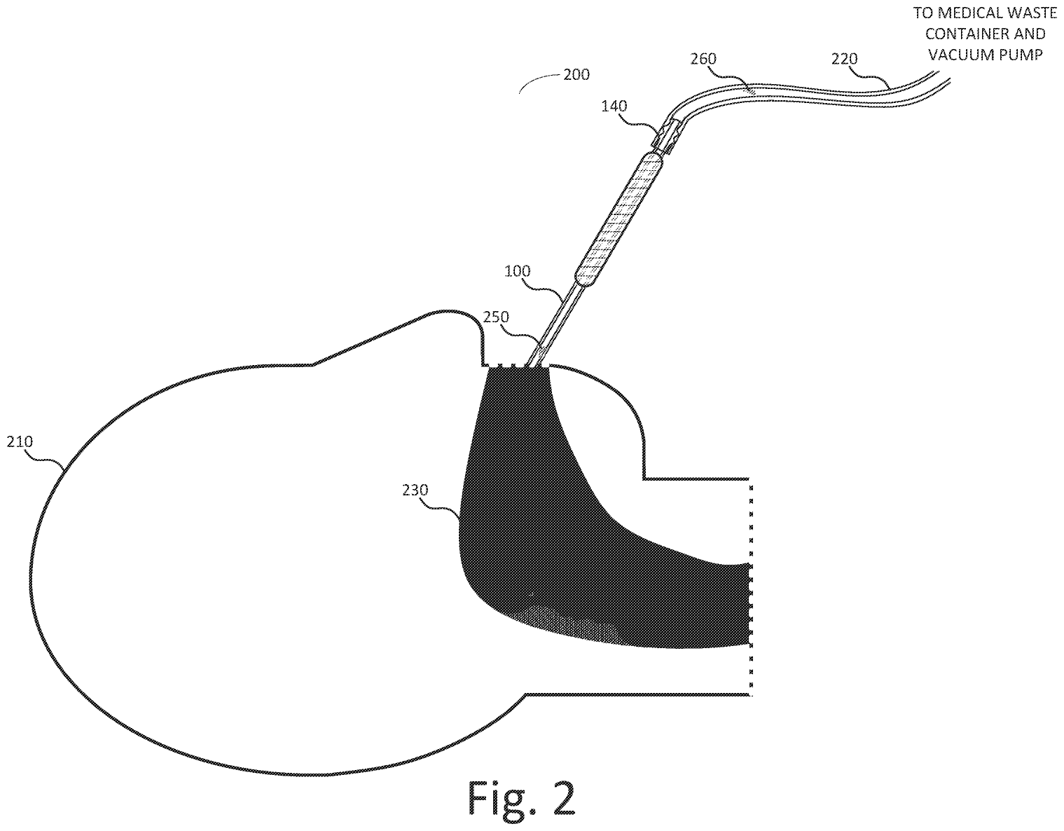

FIG. 2 illustrates a medical suction tip combined with a use indicator as it is used in a medical procedure wherein bodily secretions are evacuated from the patient, consistent with embodiments disclosed herein. The medical procedure 200 consists of a medical suction tip 100, a patient 210, and a flexible hose 220. The flexible hose connects to the grooved connecting interface 140 of the medical suction tip 100, conveying suction from the vacuum pump (not depicted) to the medical suction tip 100, and conveying an evacuation path for bodily secretions from the patient 210, through the medical suction tip 100, and out to the medical waste container (not depicted).

During the medical procedure 200, the medical suction tip 100 may be inserted into the respiratory pathway 230 of patient 210. The use indicator 150 may be located on the medical suction tip 100 and is also inserted into the respiratory pathway 230. As described above, the use indicator 150 may react with the environment present in the respiratory pathway, resulting in a detectable and/or conspicuous permanent or semi-permanent change to the use indicator 150. The medical suction tip 100 makes contact with a collection of bodily secretions 240 present in the respiratory pathway, applying suction to the collection of bodily secretions 240 until a piece of the bodily secretions is sucked into the medical suction tip 100. The evacuated bodily secretions travel through the medical suction tip 100, out through a flexible hose 220, and into a medical waste container (not depicted).

Upon completion of the medical procedure 200, there may be pieces of bodily secretions remaining in either the medical suction tip 100 or the flexible hose 220. Pieces of bodily secretions present in the medical suction tip 100, depicted as item 250 herein, may be an obvious indicator of use. However, in cases where pieces of bodily secretions only remain in the flexible hose, depicted as item 260 herein, prior usage is not as clear because such remainders of bodily secretions could be the result of a previous procedure that was done with a different medical suction tip 100. Under such circumstances, use indicator 150 provides confirmation of use, as it will have permanently or semi-permanently changed upon any use of the medical suction tip 100. Assuming this medical procedure 200 is the first usage of the medical suction tip 100, the use indicator 150 will have detectably and/or conspicuously changed after the procedure 200. Because the use indicator 150 detectably and/or conspicuously changed, a medical professional attempting to later use the medical suction tip 100 will be less likely to confuse the used medical suction tip 100 for an unused medical suction tip, and will know to engage in the appropriate disposal or sterilization procedures.

While the depicted medical procedure 200 is an evacuation of bodily secretions from the respiratory pathway 230 of patient 210. The disclosed use indicator device 150 may be applied in many different ways, in the context of many different procedures involving many different bodily environments as known in the art. For example, in some embodiments (not illustrated), the use indicator 150 may be incorporated in a Poole suction tip, reacting with blood present during an open heart surgery. In other examples (not illustrated), the use indicator 150 may be placed on a urinary catheter, reacting with urine present in the patient's urinary tract. The use indicator 150 may be combined with any device wherein the intended bodily environment presents unique characteristics that react with materials to produce detectable, conspicuous, and/or permanent or semi-permanent changes.

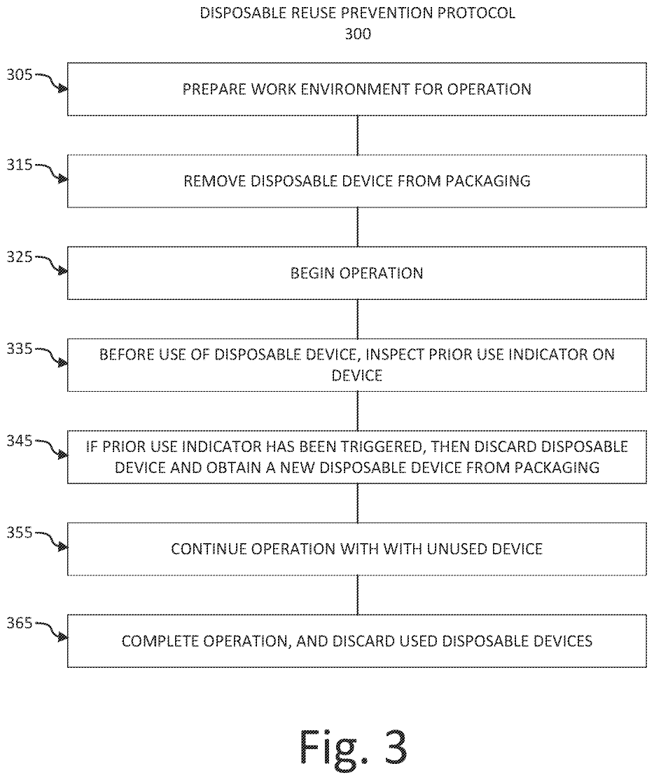

FIG. 3 is a flowchart describing a protocol for the prevention of unintentional reuse of disposable medical devices, consistent with embodiments disclosed herein. Many medical, dental, and veterinary devices are disposable or have disposable components. In order to prevent reuse of such disposables, a disposable reuse prevention protocol 300 is followed. Though the following steps are discussed in a specific order, said order is not limiting, and upon reading this disclosure, a person of ordinary skill in the art will readily imagine numerous other orderings which could be followed to prevent reuse of disposables.

As illustrated, the working environment for the medical, dental, or veterinary operation is prepared at step 305. Such preparation may include assembling the requisite tooling in a convenient location, anesthetizing the patient, and sterilizing the requisite tooling and target insertion area of the patient. Disposables may be removed from the associated packaging at step 315. Disposables may be packaged in sealed packaging that ensures the device is sterile and previously unused. The operation starts at step 325. The associated use indicator may be inspected at step 335. If the use indicator has been triggered, i.e. it has changed in appearance from how it originally came out of the packaging, the used disposable may be discarded and a new and unused disposable may be obtained from the associated packaging at step 345. The operation may continue with the unused disposable at step 355. The used disposable devices may be discarded at step 365.

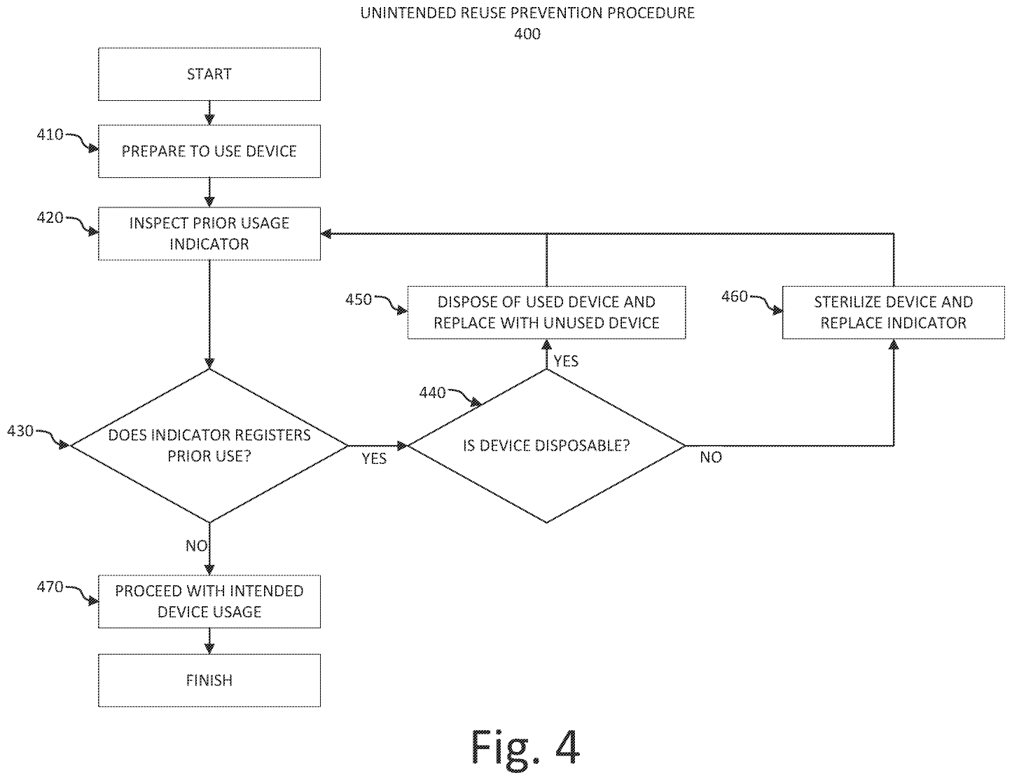

FIG. 4 illustrates a decision tree depicting a generalized procedure for preventing the unintended reuse of medical, dental, or veterinary devices, consistent with embodiments disclosed herein. The unintended reuse prevention procedure 400 is depicted as a set of steps and choices. It should be understood, that many other prevention procedures are possible using a use indicator as disclosed herein that achieve the same result with different orderings or conceptualizations of the steps.

The medical, dental, or veterinary professional prepares to use the medical, dental, or veterinary device at step 410. Such preparation may include actions such as assembling all the relevant devices in a convenient location, anesthetizing the patient, or sterilizing the relevant devices. The medical, dental, or veterinary professional inspects the use indicator at step 420. At decision point 430, the medical, dental, or veterinary professional determines if the indicator registers use. Use may be determined based on whether one of the indicator's detectable and/or conspicuous characteristics has changed from its default state corresponding to non-use. If the use indicator registers use, then the medical, dental, or veterinary professional proceeds to decision point 440, where the medical, dental, or veterinary professional determines if the device is disposable. If the device is disposable, then the medical, dental, or veterinary professional disposes of the used disposable device and replaces it with an unused disposable device or sterilized reusable device at step 450. If the device is not disposable, then the medical, dental, or veterinary professional sterilizes the device so that it can be reused, and replaces the triggered use indicator with a new use indicator that does not register a use at step 460. In relation to reusable devices, the use indicator may be reset to its default state relating to non-use, or the use indicator may be replaced with a new untriggered use indicator, immediately after sterilization and before any use. The medical, dental, or veterinary professional is returned to step 420 and must reinspect the use indicator 420. If, at decision point 430, the use indicator does not register a use, then the medical, dental, or veterinary professional is safe to proceed with the intended device usage.

The described procedure will help to ensure that medical, dental, or veterinary professionals consistently inspect a reliable metric for use (the use indicator) before using any associated device. This inspection will cut down on confusion between used devices and unused devices, resulting in the appropriate discarding of used devices and the appropriate sterilization of reusable devices, as opposed to unintentional reuse of unsanitary used devices. With the appropriate discarding or sterilization of medical, dental, and veterinary devices, the spread of infectious disease and exposure of patients to unsanitary conditions will be reduced.

As discussed previously, the use indicator described above may employ numerous different mechanisms to detect and indicate prior usage. Some of the use indicators described above may utilize logical circuits or engines. For instance, when using a system akin to the common RFID system to track usage, an associated electronic database would be created and stored on a computing device, wherein the electronic database would track exactly which devices have been used and exactly which devices have not been used.

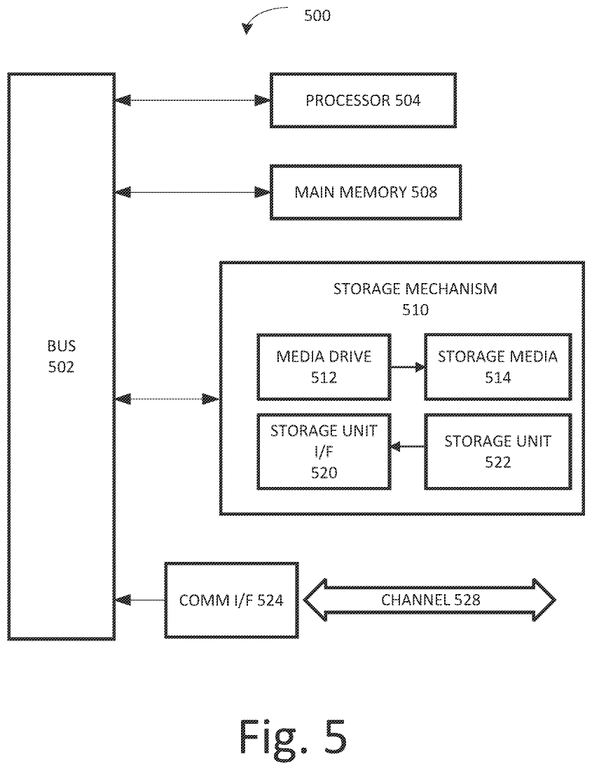

As used herein, the term engine might describe a given unit of functionality that can be performed in accordance with one or more embodiments of the technology disclosed herein. As used herein, an engine might be implemented utilizing any form of hardware, software, or a combination thereof. For example, one or more processors, controllers, ASICs, PLAs, PALs, CPLDs, FPGAs, logical components, software routines or other mechanisms might be implemented to make up a engine. In implementation, the various engines described herein might be implemented as discrete engines or the functions and features described can be shared in part or in total among one or more engines. In other words, as would be apparent to one of ordinary skill in the art after reading this description, the various features and functionality described herein may be implemented in any given application and can be implemented in one or more separate or shared engines in various combinations and permutations. Even though various features or elements of functionality may be individually described or claimed as separate engines, one of ordinary skill in the art will understand that these features and functionality can be shared among one or more common software and hardware elements, and such description shall not require or imply that separate hardware or software components are used to implement such features or functionality.

Where logical circuits or engines of the technology are implemented in whole or in part using software, in one embodiment, these software elements can be implemented to operate with a computing or processing engine capable of carrying out the functionality described with respect thereto. One such example computing engine is shown in FIG. 5. Various embodiments are described in terms of this example computing engine 500. After reading this description, it will become apparent to a person skilled in the relevant art how to implement the technology using other computing engines or architectures.

Referring now to FIG. 5, computing system 500 may represent, for example, computing or processing capabilities found within desktop, laptop and notebook computers; hand-held computing devices (PDA's, smart phones, cell phones, palmtops, etc.); mainframes, supercomputers, workstations or servers; or any other type of special-purpose or general-purpose computing devices as may be desirable or appropriate for a given application or environment. Computing engine 500 might also represent computing capabilities embedded within or otherwise available to a given device. For example, a computing engine might be found in other electronic devices such as, for example, digital cameras, navigation systems, cellular telephones, portable computing devices, modems, routers, WAPs, terminals and other electronic devices that might include some form of processing capability.

Computing system 500 might include, for example, one or more processors, controllers, control engines, or other processing devices, such as a processor 504. Processor 504 might be implemented using a general-purpose or special-purpose processing engine such as, for example, a microprocessor, controller, or other control logic. In the illustrated example, processor 504 is connected to a bus 502, although any communication medium can be used to facilitate interaction with other logical circuits of computing engine 500 or to communicate externally.

Computing system 500 might also include one or more memory engines, simply referred to herein as main memory 508. For example, preferably random access memory (RAM) or other dynamic memory, might be used for storing information and instructions to be executed by processor 504. Main memory 508 might also be used for storing temporary variables or other intermediate information during execution of instructions to be executed by processor 504. Computing engine 500 might likewise include a read only memory (“ROM”) or other static storage device coupled to bus 502 for storing static information and instructions for processor 504.

The computing system 500 might also include one or more various forms of information storage mechanism 510, which might include, for example, a media drive 512 and a storage unit interface 520. The media drive 512 might include a drive or other mechanism to support fixed or removable storage media 514. For example, a hard disk drive, a floppy disk drive, a magnetic tape drive, an optical disk drive, a CD or DVD drive (R or RW), or other removable or fixed media drive might be provided. Accordingly, storage media 514 might include, for example, a hard disk, a floppy disk, magnetic tape, cartridge, optical disk, a CD or DVD, or other fixed or removable medium that is read by, written to or accessed by media drive 512. As these examples illustrate, the storage media 514 can include a computer usable storage medium having stored therein computer software or data.

In alternative embodiments, information storage mechanism 510 might include other similar instrumentalities for allowing computer programs or other instructions or data to be loaded into computing engine 500. Such instrumentalities might include, for example, a fixed or removable storage unit 522 and an interface 520. Examples of such storage units 522 and interfaces 520 can include a program cartridge and cartridge interface, a removable memory (for example, a flash memory or other removable memory engine) and memory slot, a PCMCIA slot and card, and other fixed or removable storage units 522 and interfaces 520 that allow software and data to be transferred from the storage unit 522 to computing engine 500.

Computing engine 500 might also include a communications interface 524. Communications interface 524 might be used to allow software and data to be transferred between computing engine 500 and external devices. Examples of communications interface 524 might include a modem or softmodem, a network interface (such as an Ethernet, network interface card, WiMedia, IEEE 802.XX or other interface), a communications port (such as for example, a USB port, IR port, RS232 port Bluetooth® interface, or other port), or other communications interface. Software and data transferred via communications interface 524 might typically be carried on signals, which can be electronic, electromagnetic (which includes optical) or other signals capable of being exchanged by a given communications interface 524. These signals might be provided to communications interface 524 via a channel 528. This channel 528 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to media such as, for example, memory 508, storage unit 520, media 514, and channel 528. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing engine 500 to perform features or functions of the disclosed technology as discussed herein.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that can be included in the disclosed technology. The disclosed technology is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the technology disclosed herein. Also, a multitude of different constituent component names other than those depicted herein can be applied to the various partitions. Additionally, with regard to flow diagrams, operational descriptions, decision trees, and method claims, the order in which the steps are presented herein shall not mandate that various embodiments be implemented to perform the recited functionality in the same order unless the context dictates otherwise.

Additionally, with regard to flow diagrams, operational descriptions, decision trees, and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “component” does not imply that the components or functionality described or claimed as part of the component are all configured in a common package. Indeed, any or all of the various components of a component, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A device comprising:
a proximal end; and
a distal end configured to interact with a human or animal subject, the distal end comprising a use indication portion;
wherein the use indication portion comprises a reactive agent to react with a foreign substance to exhibit a visible change in appearance of the reactive agent.

2. The device of claim 1, further comprising a disposable Yankauer suction tip at the distal end of the device.

3. The device of claim 1, further comprising a disposable Frazier suction tip at the distal end of the device.

4. The device of claim 1, further comprising a Poole suction tip at the distal end of the device.

5. The device of claim 1, further comprising a catheter at the distal end of the device.

6. The device of claim 1, wherein the reactive agent comprises a pH indicator.

7. The device of claim 1, wherein the reactive agent is configured to reacts with a range of pH representative of saliva, blood, mucus, or urine.

8. The device of claim 1, wherein the use indicator reacts with a range of pH representative of urine.

9. The device of claim 1, wherein the reactive agent comprises a moisture or humidity.

10. The device of claim 1, further comprising a sterile package such that the device is sealed inside of the sterile package in a first configuration and removed from the sterile package in a second configuration.

11. The device of claim 1, wherein the device is a medical device.

12. The device of claim 1, further comprising a selectively detachable use indication region on the distal end, wherein the reactive agent is disposed on the use indication region.

13. The device of claim 1, wherein the distal end is configured to interact with a human patient.

14. A method for identifying whether a medical device has been used on a patient, the method comprising:
identifying, on a use indication region of a distal end of the disposable medical device, whether a reaction has occurred to change the appearance of the use indication region;
wherein the reaction comprises exposing a reagent from the use indication region to body fluid.

15. The method of claim 14, further comprising determining that the use indication region has changed color from the reaction.
a disposable Yankauer suction tip at the distal end of the device.

16. The method of claim 14, wherein the use indication region comprises a pH indicator.

17. The method of claim 14, further comprising disposing the medical device if the use indication region indicates that the medical device had been used.

18. The method of claim 14, further comprising sterilizing the medical device if the use indication region indicates that the medical device had been used.

19. The method of claim 14, wherein the medical device comprises a Yankauer suction tip at a distal end of the device.

20. The method of claim 14, wherein the medical device comprises a Poole suction tip at a distal end of the device.
